(19) United States
(12) Patent Application Publication
(10) Pub. No.: US 2009/0124012 A1
(43) Pub. Date: May 14, 2009

(54) TOXIN/ANTITOXIN SYSTEMS AND METHODS FOR REGULATING CELLULAR GROWTH, METABOLIC ENGINEERING AND PRODUCTION OF RECOMBINANT PROTEINS

(75) Inventors: Yuri NIKOLSKY, Del Mar, CA (US); Mark BAEV, Toronto (CA)

Correspondence Address: MORRISON & FOERSTER LLP, 12531 HIGH BLUFF DRIVE, SUITE 100, SAN DIEGO, CA, 92130-2040, US

(73) Assignee: Mazef Biosciences, LLC, Del Mar, CA (US)

(21) Appl. No.: 12/189,093

(22) Filed: Aug. 8, 2008

Related U.S. Application Data
(60) Provisional application No. 60/963,948, filed on Aug. 8, 2007.

(51) Int. Cl. C12N 15/82; C12N 5/06

(52) U.S. Cl. 435/455; 435/325; 435/375

(57) ABSTRACT

The present invention provides compositions and method for regulating cellular growth and metabolism, intra- and extracellular enzymatic activities, and synthesis of endogenous and/or heterologous proteins, comprising the steps of cloning genes encoding an mRNA interferase (toxin) and its cognate antitoxin; expressing these proteins in a host cell from two separate constitutive or inducible promoters on one or more plasmid vectors or on a chromosome; and regulating the cellular growth and metabolism by controlling the ratio of toxin and antitoxin present in the host cell. Optionally, the method provides further steps of modifying an endogenous or heterologous gene of interest to substitute all mRNA recognition sequences with sequences that are not cleavable by the mRNA interferase being expressed without any change in the amino acid sequence of the protein encoded by the gene; and co-expressing the gene of interest in the same host cell.

### CROSS-REFERENCE TO RELATED APPLICATIONS

This application claims benefit of priority to provisional U.S. Patent Application Ser. No. 60/963,948, filed Aug. 8, 2007, the content of which is hereby incorporated by reference in its entirety.

### FIELD OF THE INVENTION

The invention provides compositions and methods for the regulation of cellular growth, synthesis of endogenous and/or heterologous proteins, intra- and extracellular enzymatic activities, and cellular metabolism at the level of translation. In particular, it relates to regulated expression of components of toxin/antitoxin (TA) modules comprising mRNA interferases (sequence-specific endoribonucleases) such as the E. coli proteins MazF, ChpBK, PemK, a B. subtilis protein YdcE, and their cognate antitoxins such as MazE, ChpBI, PemI, and YdcD.

### BACKGROUND OF THE INVENTION

The growth rate of microbial cultures is typically regulated by external factors: either by composition of nutrient medium and the use of feeding strategies limiting cellular growth (growth limitation by substrate availability), or by changing the physicochemical conditions (e.g., by lowering the temperature). However, these are suboptimal methods. Nutrient limitation, though broadly used in the fermentation industry (Lee, High cell-density culture of Escherichia coli, Trends Biotechnol. 1996, 14:98-105; Riesenberg & Guthke, High-cell-density cultivation of microorganisms, Appl. Microbiol. Biotechnol. 1999, 51:422-430), induces both the stringent response and the general stress response in the cell (Teich, et al., Biotechnol. Prog. 1999, 15:123-129); besides the product synthesis may be limited by carbon and energy sources under these conditions. (Sanden, et al., Limiting factors in Escherichia coli fed-batch production of recombinant proteins, Biotechnol. Bioeng. 2003, 81:158-166). Cultivation at low temperatures is not always technically feasible in the industry since large-scale fermentors usually are operated at maximum cooling capacity. (Hensing, et al., Physiological and technical aspects of large-scale heterologous-protein production with yeasts, Ant. van Leeuwenhoek 1995, 67:261-279). Low temperature is also known as one of the typical environmental stress factors for microorganisms. (Mattanovich, et al., Stress in recombinant protein producing yeasts, J. Biotechnol. 2004, 113:121-135).

mRNA interferases are sequence-specific endoribonucleases which cleave mRNAs independently of ribosomes to deprive them of substrates for translation. (Zhang, et al., Insights into the mRNA cleavage mechanism by MazF, an mRNA interferase, J. Biol. Chem. 2005, 280:3143-3150). These enzymes are toxic to cells because they cause mRNA degradation, which results in the inhibition of protein synthesis and cellular growth. (Condon, C., Shutdown decay of mRNA, Mol. Microbiol. 2006, 61:573-583; Inouye, M., The discovery of mRNA interferases: implication in bacterial physiology and application to biotechnology. J. Cell. Physiol. 2006, 209:670-676).

In bacteria, mRNA interferases are co-transcribed with their cognate antitoxins in an operon. (Ruiz-Echevarria, et al., The kis and kid genes of the parD maintenance system of plasmid R1 form an operon that is autoregulated at the level of transcription by the coordinated action of the Kis and Kid proteins, Mol. Microbiol. 1991, 5:2685-2693; Tsuchimoto and Ohtsubo, Autoregulation by cooperative binding of the PemI and PemK proteins to the promoter region of the pem operon, Mol. Gen. Genet. 1993, 237:81-88; Marianovsky, et al., The regulation of the Escherichia coli mazEF promoter involves an unusual alternating palindrome, J. Biol. Chem. 2001, 276:5975-5984). Under normal growth conditions, the toxin (mRNA interferase) and antitoxin proteins are produced in a coordinated fashion and form a stable complex in the cell, and thus the toxic effect of mRNA interferases is not exerted. (Engelberg-Kulka, et al., Bacterial programmed cell death systems as targets for antibiotics, Trends Microbiol. 2004, 12:66-71.; Gerdes, et al., Prokaryotic toxin-antitoxin stress-response loci, Nat. Rev. Microbiol. 2005, 3:371-382). However, the antitoxin proteins are unstable. (Inouye (2006), see supra). Any environmental stress causing growth inhibition affects the balance between a toxin and an antitoxin, leading to the release of unbound toxins in the cell (Id.). The released toxin attacks and cleaves mRNAs, thus inhibiting protein synthesis and the cell growth. Under severe stress, which releases most toxins from their antitoxins, cell growth may be completely arrested. (Id.)

The MazE-MazF system is the best studied TA pair. MazF is a sequence-specific endoribonuclease, which cleaves mRNAs at ACA sequences. (Zhang, et al., MazF cleaves cellular mRMAs specifically at ACA to block protein synthesis in Escherichia coli, Mol. Cell 2003b, 12:913-923). MazF is a stable protein. In contrast, the MazE antitoxin is labile due to degradation by the ATP-dependent serine protease ClpA. (Aizenman, et al., An Escherichia coli chromosomal ‘addiction module’ regulated by guanosine 3′5′-bispyrophosphate: a model for programmed bacterial cell death, Proc Natl. Acad. Sci. USA 1996, 93:6059-6063). The mazEF operon is negatively autoregulated by either MazE or a MazE-MazF complex. (Marianovsky, et al. (2001) supra; Zhang, et al., Characterization of the interactions within the mazEF addiction module of Escherichia coli, J. Biol. Chem. 2003a, 278:32300-32306).

MazE-MazF-mediated cell growth arrest occurs when expression of the operon is inhibited. As a result, MazF releases from its complex with much less stable MazE. MazF activation is induced by severe amino acid or thymine starvation (Christensen, et al., Toxin-antitoxin loci as stress-response-elements: ChpAK/MazF and ChpBK cleave translated RNAs and are counteracted by tmRNA, J. Mol. Biol. 2003, 332:809-819; Sat, et al., The Escherichia coli mazEF suicide module mediates thymineless death, J. Bacteriol. 2003, 185:1803-1807, Sat, et al., Programmed cell death of Escherichia coli: some antibiotics can trigger mazEF lethality, J. Bacteriol. 2001, 183:2041-2045), high temperature, oxidative stress, UV irradiation (Hazan, et al., Escherichia coli mazEF-mediated cell death is triggered by various stressful conditions, J. Bacteriol. 2004, 186:3663-3669). Overproduction of MazF in plasmid-transformed E. coli cells completely inhibits cellular growth as a result of degradation of almost all cellular mRNAs and inhibition of protein synthesis. (Christensen, et al. (2003), supra; Zhang, et al. (2003b), supra). However, MazF induction does not affect DNA and RNA synthesis, which indicates that metabolic activities necessary for ATP production and nucleotide biosynthesis are retained in the cells overproducing MazF. This physiological state has been labeled as “quasi-dormancy”. (Suzuki, et al., Single protein production in living cells facilitated by an mRNA interferase, Mol. Cell 2005, 18:253-261). Subsequent overexpression of MazE restores the protein synthesis in the cell. (Christensen, et al. (2003), supra).

E. coli cells transformed to overproduce MazF are able to produce only those proteins whose genes have been engineered to alter all the recognition ACA sequences in the corresponding mRNAs to non-MazF-cleavable sequences. This modification does not affect the amino acid sequence of the synthesized proteins due to the general codon degeneracy. (Suzuki, et al. (2005), supra). Several prokaryotic and eukaryotic proteins have been expressed in quasi-dormant E. coli cells, including human eotaxin, yeast protein Hsp10, RNA polymerase subunit Rpb12, E. coli proteins EnvZB and ScpA, and the lipoprotein signal peptidase LspA. (Id.). These recombinant proteins were produced at a level of up to 30% of the total cellular protein with no background cellular protein synthesis. The quasi-dormant cells were metabolically active and synthesized the recombinant proteins for more than seven days. This approach to protein biosynthesis has been labeled the “Single Protein Production (SPP)” system. (Id.)

Despite obvious merits, the SPP system has limited industrial applications. It can be used, for instance, for manufacturing small amounts of expensive proteins labeled with radioactive isotopes such as N15 and C13 or toxic amino acid analogs without labeling any other cellular proteins. (Id.). The MazF over-expression from a strong promoter used in the SPP system results in a complete shutdown of protein synthesis and arrest of cell growth, whereas the conventional bio-manufacturing is mainly based on the use of growing cells.

### SUMMARY OF THE INVENTION

The present invention provides improved systems for the internal regulation of the growth rate of cells and for the purposes of metabolic engineering and industrial strain development.

The present invention provides methods for regulating cellular growth and metabolism, intra- and extracellular enzymatic activities, and synthesis of endogenous and/or heterologous proteins, comprising the steps of cloning genes encoding one or more mRNA interferases (sequence-specific endoribonucleases, which can be a toxin) and its cognate antitoxin; expressing these proteins from separate constitutive or inducible promoters on one or more plasmid vectors or on a chromosome; and regulating the cellular growth and metabolism by controlling the ratio of toxin and antitoxin present in the cell.

In one aspect, the method of the present invention provides a further step of modifying an endogenous gene to introduce mRNA recognition nucleotide sequences cleavable by the mRNA interferase being expressed without any change in the amino acid sequence of the protein encoded by the gene.

In another aspect of the present invention, the method provides further steps of modifying an endogenous or heterologous gene of interest to substitute all mRNA recognition nucleotide sequences (cleavable by the mRNA interferase) with sequences that are not cleavable by the mRNA interferase being expressed without any change in the amino acid sequence of the protein encoded by the gene and co-expressing the gene of interest in the same host cell.

### BRIEF DESCRIPTION OF THE DRAWINGS

FIGS. 1A and 1B illustrate expression profiles of mazE and mazF during growth of E. coli strain MG1655 on LB medium. FIG. 1A shows the optical density of bacterial culture as a function of time. FIG. 1B shows relative expression levels of MazF and MazE (right axis) and the growth rate μ (left axis), which is determined as follows: μ=(ln[X]−ln[X₀])/T, where T is time, and X₀ and X are optical densities of the bacterial culture at time points zero and T, respectively.

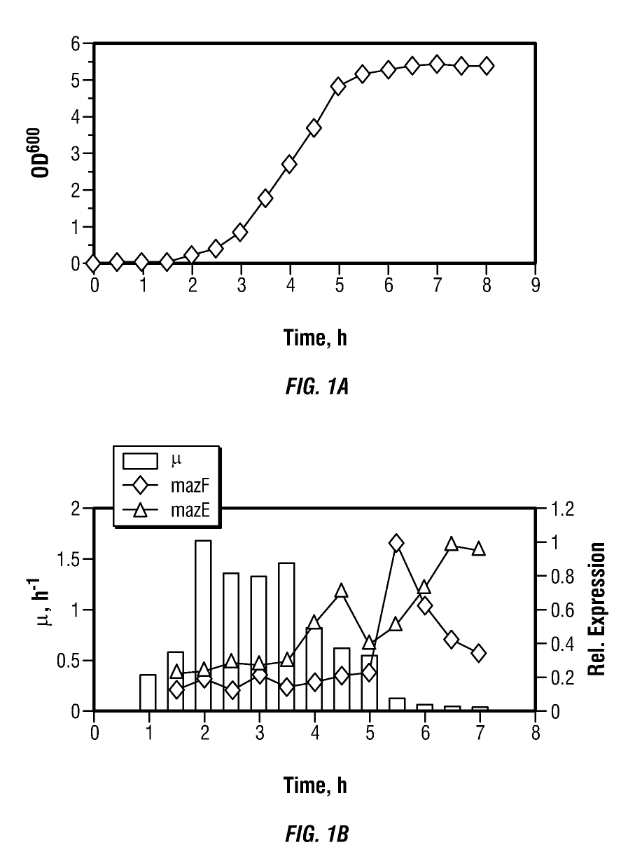

FIG. 2 shows the pKD20 “helper” vector carrying three genes of the Red recombinase system, used for MazE and MazF cloning in Escherichia coli.

FIG. 3 shows pRS306/316 vectors used for MazE and MazF cloning in Saccharomyces cerevisiae.

FIGS. 4A and 4B demonstrate the feasibility of regulating the induction of a bacterial promoter at the mRNA level by varying the concentration of inducer (IPTG) (see Epshtein V. & Nudler E., Cooperation between RNA polymerase molecules in transcription elongation, Science 2005, 300:801-805).The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DETAILED DESCRIPTION OF THE INVENTION

The present invention provides compositions and methods for regulating cellular growth and metabolism, intra- and extracellular enzymatic activities, and synthesis of endogenous and/or heterologous proteins, comprising the steps of cloning genes encoding an mRNA interferase (toxin) and its cognate antitoxin; expressing these proteins from separate constitutive or inducible promoters on one or more plasmid vectors or on a chromosome; and regulating the cellular growth and metabolism by controlling the ratio of toxin and antitoxin present in the cell.

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are incorporated herein by reference, the definition set forth in this section prevails over the definition that is incorporated herein by reference.

Citation of publications or documents is not intended as an admission that any of such publications or documents are pertinent prior art, nor does it constitute any admission as to the contents or date of these publications or documents.

As used herein, “a” or “an” means “at least one” or “one or more.”

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. The phrases “nucleic acid” or “nucleic acid sequence” includes oligonucleotide, nucleotide, polynucleotide, or to a fragment of any of these, to DNA or RNA (e.g., mRNA, rRNA, tRNA, iRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., e.g., double stranded iRNAs, e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. “Oligonucleotide” includes either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide can ligate to a fragment that has not been dephosphorylated.

A “coding sequence of” or a “nucleotide sequence encoding” a particular polypeptide or protein, is a nucleic acid sequence which is transcribed and translated into a polypeptide or protein when placed under the control of appropriate regulatory sequences.

The term “gene” means the segment of DNA involved in producing a polypeptide chain; it includes regions preceding and following the coding region (leader and trailer) as well as, where applicable, intervening sequences (introns) between individual coding segments (exons). “Operably linked” as used herein refers to a functional relationship between two or more nucleic acid (e.g., DNA) segments. Typically, it refers to the functional relationship of transcriptional regulatory sequence to a transcribed sequence. For example, a promoter is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “expression cassette” as used herein refers to a nucleotide sequence which is capable of affecting expression of a structural gene (i.e., a protein coding sequence, such as MazF and MazE) in a host compatible with such sequences. Expression cassettes include at least a promoter operably linked with the polypeptide coding sequence; and, in one aspect, with other sequences, e.g., transcription termination signals. Additional factors necessary or helpful in effecting expression may also be used, e.g., enhancers. Thus, expression cassettes also include plasmids, expression vectors, recombinant viruses, any form of recombinant “naked DNA” vector, and the like.

A “vector” comprises a nucleic acid that can infect, transfect, transiently or permanently transduce a cell. It will be recognized that a vector can be a naked nucleic acid, or a nucleic acid complexed with protein or lipid. The vector in one aspect comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

As used herein, the term “promoter” includes all sequences capable of driving transcription of a coding sequence in a cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

As used herein, the term “isolated” means that the material (e.g., a nucleic acid, a polypeptide, a cell) is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition and still be isolated in that such vector or composition is not part of its natural environment.

As used herein, the term “purified” does not require absolute purity; rather, it is intended as a relative definition. Individual nucleic acids obtained from a library have been conventionally purified to electrophoretic homogeneity. The sequences obtained from these clones could not be obtained directly either from the library or from total human DNA. The purified nucleic acids of the invention have been purified from the remainder of the genomic DNA in the organism by at least 10⁴-10⁶ fold. However, the term “purified” also includes nucleic acids that have been purified from the remainder of the genomic DNA or from other sequences in a library or other environment by at least one order of magnitude, typically two or three orders and more typically four or five orders of magnitude.

As used herein, the term “recombinant” means that the nucleic acid is adjacent to a “backbone” nucleic acid to which it is not adjacent in its natural environment. Additionally, to be “enriched” the nucleic acids will represent 5% or more of the number of nucleic acid inserts in a population of nucleic acid backbone molecules. Backbone molecules according to the invention include nucleic acids such as expression vectors, self-replicating nucleic acids, viruses, integrating nucleic acids and other vectors or nucleic acids used to maintain or manipulate a nucleic acid insert of interest. Typically, the enriched nucleic acids represent 15% or more of the number of nucleic acid inserts in the population of recombinant backbone molecules. More typically, the enriched nucleic acids represent 50% or more of the number of nucleic acid inserts in the population of recombinant backbone molecules. In a one aspect, the enriched nucleic acids represent 90% or more of the number of nucleic acid inserts in the population of recombinant backbone molecules.

“Plasmids” are designated by a lower case “p” preceded and/or followed by capital letters and/or numbers. The starting plasmids herein are either commercially available, publicly available on an unrestricted basis, or can be constructed from available plasmids in accord with published procedures. In addition, equivalent plasmids to those described herein are known in the art and will be apparent to the ordinarily skilled artisan. “Plasmids” can be commercially available, publicly available on an unrestricted basis, or can be constructed from available plasmids in accord with published procedures. Equivalent plasmids to those described herein are known in the art and will be apparent to the ordinarily skilled artisan.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I. A promoter sequence is “operably linked to” a coding sequence when RNA polymerase which initiates transcription at the promoter will transcribe the coding sequence into mRNA. Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used. Promoters suitable for expressing the polypeptide or fragment thereof in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK) and the acid phosphatase promoter. Fungal promoters include the V factor promoter. Eukaryotic promoters include the CMV immediate early promoter, the HSV thymidine kinase promoter, heat shock promoters, the early and late SV40 promoter, LTRs from retroviruses and the mouse metallothionein-I promoter. Other promoters known to control expression of genes in prokaryotic or eukaryotic cells or their viruses may also be used.

Expression Vectors and Cloning Vehicles

The invention provides expression vectors and cloning vehicles comprising nucleic acids of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector can comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells can also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

The vector can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

The appropriate DNA sequence may be inserted into the vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley 503 Sons, Inc. 1997 and Sambrook et al., Molecular Cloning: A Laboratory Manual 2nd Ed., Cold Spring Harbor Laboratory Press (1989. Such procedures and others are deemed to be within the scope of those skilled in the art.

Host Cells and Transformed Cells

The invention also provides transformed cells comprising a nucleic acid sequence of the invention, e.g., a sequence encoding a xylanase, a mannanase and/or a glucanase of the invention, or a vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include any species within the genera Escherichia, Bacillus, Streptomyces, Salmonella, Pseudomonas and Staphylococcus, including, e.g., Escherichia coli, Lactococcus lactis, Bacillus subtilis, Bacillus cereus, Salmonella typhimurium, Pseudomonas fluorescens. Exemplary fungal cells include any species of Aspergillus. Exemplary yeast cells include any species of Pichia, Saccharomyces, Schizosaccharomyces, or Schwanniomyces, including Pichia pastoris, Saccharomyces cerevisiae, or Schizosaccharomyces pombe. Exemplary insect cells include any species of Spodoptera or Drosophila, including Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

In one aspect, the nucleic acids or vectors of the invention are introduced into the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.).

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

The nucleic acids of the invention can be expressed, or overexpressed, in any in vitro or in vivo expression system. Any cell culture systems can be employed to express, or over-express, recombinant protein, including bacterial, insect, yeast, fungal or mammalian cultures. Over-expression can be effected by appropriate choice of promoters, enhancers, vectors (e.g., use of replicon vectors, dicistronic vectors (see, e.g., Gurtu (1996) Biochem. Biophys. Res. Commun. 229:295-8), media, culture systems and the like. In one aspect, gene amplification using selection markers, e.g., glutamine synthetase (see, e.g., Sanders (1987) Dev. Biol. Stand. 66:55-63), in cell systems are used to overexpress the polypeptides of the invention.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

As discussed above, the present invention provides methods for regulating cellular growth and metabolism, intra- and extracellular enzymatic activities, and synthesis of endogenous and/or heterologous proteins, comprising the steps of cloning genes encoding one or more mRNA interferases (sequence-specific endoribonucleases, which can be a toxin) and its cognate antitoxin; expressing these proteins from separate constitutive or inducible promoters on one or more plasmid vectors or on a chromosome; and regulating the cellular growth and metabolism by controlling the ratio of toxin and antitoxin present in the cell.

In one aspect, the method of the present invention optionally provides a further step of modifying an endogenous gene to introduce mRNA recognition nucleotide sequences cleavable by the mRNA interferase being expressed without any change in the amino acid sequence of the protein encoded by the gene.

In another aspect of the present invention, the method optionally provides further steps of modifying an endogenous or heterologous gene of interest to substitute all mRNA recognition nucleotide sequences cleavable by the mRNA interferase with sequences that are not cleavable by the mRNA interferase being expressed without any change in the amino acid sequence of the protein encoded by the gene and co-expressing the gene of interest in the same host cell.

Thus, the present invention provides a compositions and methods for industrial strain development. The compositions and methods as provided herein allow the manipulation of cellular metabolism at the level of translation in order to redirect metabolic fluxes to enhance production of a targeted cellular metabolite or to decrease accumulation of undesired by-products. Toxin/antitoxin (TA) tools may be generated by routine, traditional methods of recombinant DNA technology, which are well known in the art. It includes, for example, expression of MazE and MazF from different constitutive and/or inducible promoters on plasmid vectors and chromosome, engineering recombinant proteins so they lack mRNA interferase recognition sequences, and small-scale fermentation.

The compositions and methods as provided herein can attenuate expression of mRNA interferases such as, for example, MazF, ChpBK, PemK, or YdcE, by the use of a weak constitutive promoter, or can balancing expression by a simultaneous over-expression of the cognate antitoxin (MazE, ChpBI, PemI, YdcD, respectively) from a separate constitutive or inducible promoter, which results in decreasing intracellular concentration of unbound mRNA interferase molecules to the levels which are able to decrease protein synthesis in the cell, but not sufficient to arrest it completely. In one embodiment, the ratio of toxin/antitoxin intracellular concentrations are used as a factor for regulating protein synthesis and cellular metabolism at the level of mRNA translation.

The ability of the engineered TA systems of the invention to regulate, but not fully arrest, protein synthesis and metabolism is key factor for a number of applications of TA systems in industrial fermentation, strain development and metabolic engineering. In one embodiment, TA systems of the invention are used for regulation of a host strain's growth rate, which is one of important parameters of fermentation processes. For example, TA systems of the invention can be used to attenuate accumulation of acetate, a toxic metabolite, in a fermentation broth, where acetate accumulation can be a problem at high growth rates. In one embodiment, TA systems of the invention can be used to attenuate increased oxygen consumption and heat generation, which also can be a problem in cell systems with high growth rates. In alternative embodiments, TA systems of the invention are used to control all of these factors, thus allowing significant increases in the efficiency and cost-effectiveness of an industrial fermentation process.

In one embodiment, use of the mRNA interferase toxin/antitoxin systems as provided herein in host cells allows cultivating them with a predetermined lower specific growth rate under optimal growth conditions with no external limitations, i.e., under conditions of an extended unlimited growth. Moreover, in alternative embodiments, use of compositions and methods as provided herein effectively enables the regulation and fine-tuning of cell growth, which provides additional benefits for heterologous protein production such as, for example, the production of proteins that are toxic to the host cell.

In one embodiment, expression of endogenous or heterologous genes are modified such that they are no longer targeted by interferase enzymes, e.g., are modified such that they are transcribed into “non-cleavable-by-mRNA-interferase” mRNAs. This allows the cells to redirect cellular resources to the synthesis of these proteins without complete arrest of cell growth. In one embodiment, the same alteration of genes coding for key rate-limiting enzymes of particular metabolic pathways is made; this makes these pathways insensitive to the inhibition by an mRNA interferase. In one embodiment, “metabolic” genes are altered to transcribe into one or more cleavage-sequence-enriched mRNAs (e.g., ACA, in the case of MazF); this would make synthesis of the corresponding enzymes more susceptible to the inhibition by mRNA interferase, and suppress functioning of the metabolic pathway consisting of these enzymes.

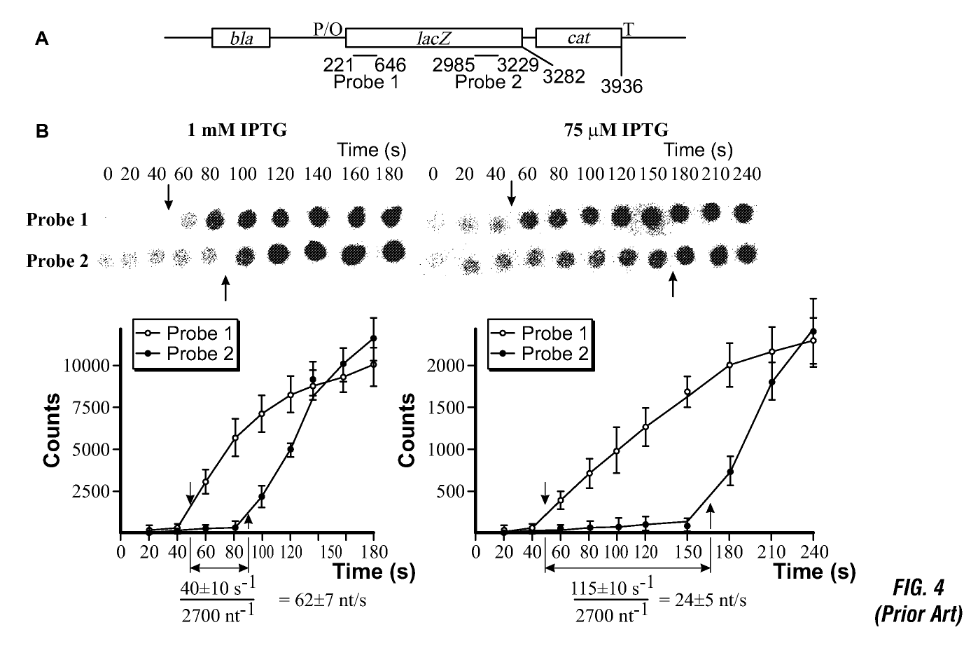

In one embodiment, both mRNA interferase toxin and antitoxin genes can be expressed under inducible conditions and independently from each other. The feasibility of regulating the induction of a bacterial promoter at the mRNA level by varying the concentration of inducer (IPTG) is known in the art. (See FIG. 4; Epshtein V. & Nudler E., Cooperation between RNA polymerase molecules in transcription elongation, Science 2005, 300:801-805). The inducible expression enables one to control the ratio of intracellular concentrations of these proteins in the course of fermentation and, by so doing, to modulate the cellular growth rate, the rate of protein synthesis, or intracellular metabolic fluxes. The mRNA interferase/antitoxin systems of the invention can be functional in virtually any living cell (both prokaryotic and eukaryotic) because of the universality of the genetic code. For example, the MazF RNA interferase expressed in human T-Rex-293 cells inhibited protein synthesis by cleavage of cellular mRNA and induced apoptosis (Zhang, et al. (2007), supra).

In one aspect, the compositions and methods of the present invention provide new fermentation technologies and optimization of existing fermentation technologies for the production of biopharmaceuticals such as, for example, erythropoietin, insulin, blood clotting factor, interferons, human growth hormone, somatotropin, tissue plasminogen activator, interleukin, hirudin, anti-hemophilia factor, human parathyroid hormone, epidermal growth factor and other growth factors, therapeutic monoclonal antibodies, and various therapeutic vaccines.

In another aspect, the compositions and methods of the present invention provide new fermentation technologies and optimization of existing fermentation technologies for the production of enzymes such as, for example, chymosin, trypsin, aspartic proteinase, serine proteases, alkaline proteases, esterases, chitinases, tannase, nitrile hydratase, streptokinase, levansucrases, xylanases, cellulases, glucoamylase, alkaline amylases, lipases, pectinases, α-amylase, pullulanase, glucose isomerase, pectate lyase, mannanase, β-glucanase, and keratinase.

In yet another aspect, the compositions and methods of the present invention provide new fermentation technologies and optimization of existing fermentation technologies for the production of antibiotics such as, for example, actinomycin, bleomycin, rifamycin, chloramphenicol, tetracycline, lincomycin, erythromycin, streptomycin, cyclohexamide, puromycin, cycloserine, bacitracin, penicillin, cephalosporin, vancomycin, polymyxin, and gramicidin.

In yet another aspect, the compositions and methods of the present invention provide new fermentation technologies and optimization of existing fermentation technologies for the production of biosurfactants such as, for example, rhamnolipids, sophorolipids, glycolipids, and lipopeptides.

In yet another aspect, the compositions and methods of the present invention provide new fermentation technologies and optimization of existing fermentation technologies for the production of biological fuels such as, for example, bioethanol, biodiesel, and biobutanol.

In yet another aspect, the compositions and methods of the present invention provide new fermentation technologies and optimization of existing fermentation technologies for the production of amino acids such as, for example, L-glutamate, L-lysine, L-phenylalanine, L-aspartic acid, L-isoleucine, L-valine, L-tryptophan, L-proline (hydroxyproline), L-threonine, L-methionine, and D-p-hydroxyphenylglycine.

In yet another aspect, the compositions and methods of the present invention provide new fermentation technologies and optimization of existing fermentation technologies for the production of organic acids such as, for example, citric acid, lactic acid, gluconic acid, acetic acid, propionic acid, succinic acid, fumaric acid, and itaconic acid.

In yet another aspect, the compositions and methods of the present invention provide new fermentation technologies and optimization of existing fermentation technologies for the production of fatty acids such as, for example, arachidonic acid, polyunsaturated fatty acid (PUBA), and γ-linoleic acid.

In yet another aspect, the compositions and methods of the present invention provide new fermentation technologies and optimization of existing fermentation technologies for the production of polyols such as, for example, glycerol, mannitol, erythritol, and xylitol.

In yet another aspect, the compositions and methods of the present invention provide new fermentation technologies and optimization of existing fermentation technologies for the production of flavors and fragrances such as, for example, vanillin, benzaldehyde, dixydroxyacetone, 4-(R)-decanolide, and 2-actyl-1-pyrroline.

In yet another aspect, the compositions and methods of the present invention provide new fermentation technologies and optimization of existing fermentation technologies for the production of nucleotides such as, for example, 5′-guanylic acid and 5′-inosinic acid.

In yet another aspect, the compositions and methods of the present invention provide new fermentation technologies and optimization of existing fermentation technologies for the production of vitamins such as, for example, vitamin C, vitamin F, vitamin B2, provitamin D2, vitamin B12, folic acid, nicotinamide, biotin, 2-keto-L-gulonic acid, and provitamin Q10.

In yet another aspect, the compositions and methods of the present invention provide new fermentation technologies and optimization of existing fermentation technologies for the production of pigments such as, for example, astaxathin, β-carotene, leucopene, monascorubrin, and rubropunctatin.

In yet another aspect, the compositions and methods of the present invention provide new fermentation technologies and optimization of existing fermentation technologies for the production of sugars and polysaccharides such as, for example, ribose, sorbose, xanthan, gellan, and dextran.

In yet another aspect, the compositions and methods of the present invention provide new fermentation technologies and optimization of existing fermentation technologies for the production of biopolymers and plastics such as, for example, polyhydroxyalkanoates (PHA), poly-γ-glutamic acid, and 1,3-propanediol.

The following examples and preparations are intended to illustrate the invention but are not intended to limit its scope. Parts and percentages are by weight unless otherwise indicated.

### EXAMPLES

### Example 1

### Baseline Growth of E. coli Strain BL21 (Low Glucose)

The following example demonstrates use of exemplary compositions and methods of the invention for the regulation of cellular growth, synthesis of endogenous and/or heterologous proteins.

Several colonies of Escherichia coli strain BL21 grown on agarized LB medium at 37° C. were inoculated into 20 ml of M9 medium supplemented with 2 g/L glucose and incubated at 220 rpm, 37° C. overnight. 2 ml of the resulting culture was used as inoculation material to start batch fermentation in a laboratory-scale fermentor with a 2L working volume. The following chemically defined growth medium was used: glucose, 4 g/L; NH₄Cl, 1.0 g/L; KH₂PO₄, 3.0 g/L; MgSO₄.7H₂O, 0.6 g/L; CaCl₂, 0.01 g/L; FeSO₄, 0.02 g/L; citric acid, 0.3 g/L; and trace metal solution, 1.9 ml/L. The trace metal solution contained: Al₂(SO₄)₃.7H₂O, 10 mg/L; CoCl₂.6H₂O, 8 mg/L; CuSO₄.H₂O, 2 mg/L; H₃BO₃, 1 mg/L; MnCl₂.4H₂O, 20 mg/L; NiCl₂.6H₂O, 1 mg/L; Na₂MoO₄.2H₂O, 5 mg/L; and ZnSO₄.7H₂O, 5 mg/L.

The pH was maintained at 6.95 automatically by titration with 5% NaOH solution. The level of oxygen in the culture was maintained at 40% saturation automatically by varying speed of impeller rotation. The temperature was maintained at 37° C. The culture growth (OD₆₀₀) was monitored spectrophotometrically. The dry cell weight (DCW) concentration was determined gravimetrically using 0.2 μm membrane filters. The concentrations of residual glucose and accumulating acetate were determined using the corresponding Enzymatic BioAnalysis kits (R-Biopharm).

The culture grew exponentially with a maximum growth rate (μₘₐₓ) of 0.81 h⁻¹. The growth stopped after about 6.5 hours. The final biomass concentration was 2.1 g(DCW)/L, which corresponds to a biomass yield on glucose 0.52 g/g. Maximum acetate accumulation of 0.52 g/L was observed at about 6 hours of fermentation, after which it started to decrease.

### Example 2

### Baseline Growth of E. coli Strain BL21 (Medium Glucose)

In a related experiment, Escherichia coli strain BL21 was grown as a batch culture on the agarized LB medium under the growth conditions as described in Example 1, with the exception that the concentration of glucose in the medium was increased to 12 g/L.

The culture grew exponentially with a maximum growth rate (μₘₐₓ) of 0.77 h⁻¹. The growth stopped after about 9 hours. The final biomass concentration was 5.9 g(DCW)/L, which corresponds to a biomass yield of 0.49 g/g. Maximum acetate accumulation of 1.1 g/L was observed at about 8 hours of fermentation, after which it started to decrease.

### Example 3

### Baseline Growth of E. coli Strain BL21 (High Glucose)

In another related experiment, Escherichia coli strain BL21 was grown as a batch culture on the agarized LB medium under the growth conditions as described in Example 1, with the exception that the concentration of glucose in the medium was increased to 24 g/L.

The culture grew exponentially with a maximum growth rate (μₘₐₓ) of 0.65 h⁻¹. The growth stopped after about 11 hours. The final biomass concentration was 10.3 g(DCW)/L which corresponds to a biomass yield of 0.43 g/g. Maximum acetate accumulation of 1.8 g/L was observed at about 10 hours of fermentation, after which it started to decrease.

Based on the results observed at the three different glucose concentrations, it is apparent the biomass yield decreases with increasing glucose concentration.

### Example 4

### Generation of Mutant MazF/MazE Bacterial Strains

In E. coli, mazF is expressed from a weak constitutive promoter, whereas the expression of mazE is knocked out. This is achieved by applying a one-step technology of gene replacement and chromosome cloning in E. coli (see, e.g., Datsenko & Wanner (2000)). The integrative low copy number pkD46 plasmid is used for the deletion of endogenous MazE and the exchange of the endogenous chromosomal MazF for a MazF copy expressed from a mutant ara BAD promoter. MazF is induced using different concentrations of arabinose added to the cultural media.

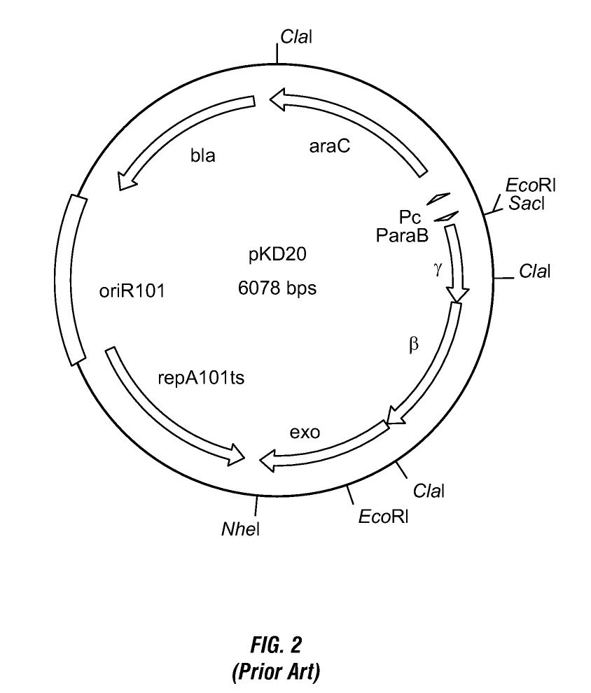

For MazE deletion, PCR fragments are generated using 70 bp oligonucleotides primers consisting of 50 bp from the promoter and non-coding (after stop-codon) region of MazE and 20 bp from the template pKD13 plasmid sequence. The host strain is simultaneously transformed with the PCR fragments and the helper plasmid pKD20 (as shown on FIG. 2) carrying three genes of the Red recombinase system. The plasmid fragment includes kan (Kanamycin resistance gene) as the selection marker and flanking FRT sequences for the follow-up excision of kan. The PCR fragments are transformed into wild type BL21 strain, and kanamycin-resistant colonies are selected, followed by DNA sequencing to select bacterial clones having MazE deletions.

Other cloning experiments with introduction of different promoters for the MazE and MazF genes can be conducted in substantially the same fashion.

### Example 5

### Growth of Mutant E. coli Constitutively Expressing MazF Under Control of a Weak Promoter and not Expressing MazE (Low Glucose)

Mutant Escherichia coli strain BL21 generated as described in Example 4 (i.e., lacking MazE and expressing MazF under the direction of a weak constitutive promoter) is grown as a batch culture on the agarized medium under the low glucose growth conditions as those described in Example 1.

The culture grows exponentially with a maximum growth rate (μₘₐₓ) of about 0.16 h⁻¹. Growth stops after about 26 hours. The final biomass concentration is about 2.1 g/L, which corresponds to a biomass yield of about 0.52 g(DCW)/g. No acetate accumulation is detected throughout the growth.

### Example 6

### Growth of Mutant E. coli Constitutively Expressing MazF Under Control of a Weak Promoter and not Expressing MazE (High Glucose)

Mutant Escherichia coli strain BL21 generated as described in Example 4 (i.e., lacking MazE and expressing MazF under the direction of a weak constitutive promoter) is grown as a batch culture on the agarized medium under the high glucose growth conditions as those described in Example 3.

The culture grows exponentially with a maximum growth rate (μₘₐₓ) of about 0.17 h⁻¹. The growth stops after about 38 h. The final biomass concentration is about 12.3 g(DCW)/L, which corresponds to a biomass yield of about 0.51 g/g. No acetate accumulation is detected throughout the growth.

Accordingly, in contrast to the wild-type BL21 E. coli strain, the high glucose culture of the mutant BL21 E. coli strain is characterized by prolonged cell growth without a detectable accumulation of acetate or a significant loss in the biomass yield.

### Example 7

### Growth of Mutant E. coli Constitutively Expressing MazF Under Control of a Weak Constitutive Promoter and Expressing MazE Under Control of an Inducible Promoter (Early MazE Induction)

Mutant Escherichia coli strain BL21 is generated as described in Example 4, such that MazF is expressed from a weak constitutive promoter and MazE is expressed from an inducible promoter. Induction of the MazE expression coincides with the start of fermentation. The mutant E. coli BL21 strain is grown as a batch culture on the agarized medium under the high glucose growth conditions as described in Example 3.

The culture grows exponentially with a maximum growth rate (μₘₐₓ) of about 0.62 h⁻¹. The growth stops approximately 11 h after the induction of MazE expression. The final biomass concentration is about 10.2 g/L which corresponds to a biomass yield of about 0.43 g/g. A maximum acetate accumulation of about 1.9 g/L was observed at about 10 hours of fermentation, after which it starts to decrease.

### Example 8

### Growth of Mutant E. coli Constitutively Expressing MazF Under Control of a Weak Constitutive Promoter and Expressing MazE Under Control of an Inducible Promoter (Delayed MazE Induction)

Mutant Escherichia coli strain BL21 is generated as described in Example 4, such that MazF is expressed from a weak constitutive promoter and MazE is expressed from an inducible promoter. Induction of the MazE expression is initiated about 8 hours after the start of fermentation. The mutant E. coli BL21 strain is grown as a batch culture on the agarized medium under the high glucose growth conditions as described in Example 3.

The culture grows exponentially with a maximum growth rate (μₘₐₓ) of about 0.15 h⁻¹ for the first 8 hours of fermentation and reaches an OD₆₀₀ of about 0.6, at which point an inducer is added to the culture to initiate the expression of MazE. Consequently, the growth rate increases to about 0.54 h⁻¹ within the next 1.5 hours. The growth stops approximately 8 hours after the induction. The final biomass concentration is about 10.5 g/L, which corresponds to a biomass yield of about 0.44 g/g. A maximum acetate accumulation of about 1.7 g/L is observed at about 19 hours of fermentation, after which it starts to decrease.

### Example 9

### Baseline Growth of Wild-Type S. cerevisiae Strain

Several colonies of a wild-type Saccharomyces cerevisiae strain grown on agarized YPD medium at 30° C. are inoculated into 50 ml of YPD medium and incubated at 220 rpm, 30° C. overnight. 40 ml of the resulting culture is used as inoculation material to start batch fermentation in a laboratory-scale fermentor with a 2L working volume. The following chemically defined growth medium is used: glucose, 20 g/L; (NH₄)₂SO₄, 10 g/L; KH₂PO₄, 6 g/L; MgSO₄.7H₂O, 1 g/L; EDTA, 30 mg/L; ZnSO₄.7H₂O, 9 mg/L; CoCl₂.6H₂O, 0.6 mg/L; MnCl₂.4H₂O, 2 mg/L; CuSO₄.5H₂O, 0.6 mg/L; CaCl₂.2H₂O, 9 mg/L; FeSO₄.7H₂O, 6 mg/L; NaMoO₄.2H₂O, 0.8 mg/L; H₃BO₃, 2 mg/L; KI, 0.2 mg/L; biotin, 0.10 mg/L; calcium pantothenate, 2 mg/L; nicotinic acid, 2 mg/L; inositol, 50 mg/L; thiamine HCl, 2 mg/L; pyridoxine HCl, 2 mg/L; and para-aminobenzoic acid, 0.4 mg/L.

The pH is maintained at 5.5 automatically by titration with 5% NaOH solution. The oxygen level in the culture is maintained at 40% saturation automatically by varying the speed of impeller rotation. The temperature is maintained at 30° C. The culture growth (OD₆₆₀) is monitored spectrophotometrically. The culture grows exponentially with a maximum growth rate (μₘₐₓ) of about 0.35 h⁻¹.

### Example 10

### Generation of Mutant MazF/MazE S. cerevisiae Strains

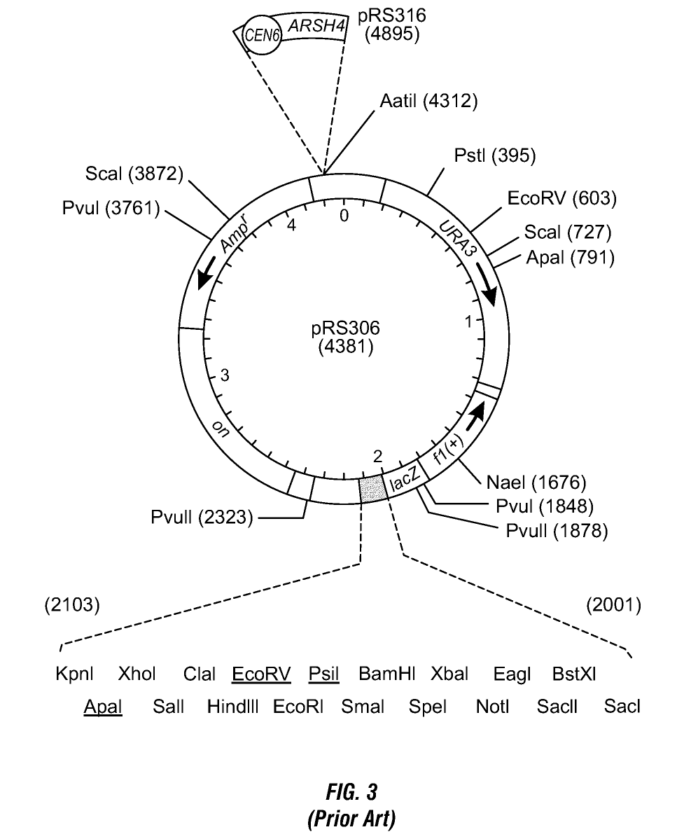

The MazE and MazF genes cloned from E. coli are expressed in haploid and diploid strains of the yeast S. cerevisiae. Two types of “shuttle” vectors of the pRS series are used for cloning. The integrative plasmid pRS306 (see FIG. 3; Sikorski & Hieter (1989)) with both yeast (CEN) and E. coli (pUC) origins of replication allows to insert the cloned genes (MazE and MazF) into the yeast genome by homologous recombination. The W303 strain of S. cerevisiae with the deletion of URA3 gene (ura 3) is used. The cloned genes integrate into yeast chromosomes at the site of deleted genes (URA3) and selected by growth on minimal media with urine as selectable marker. Consequently, the cloned genes are present in the yeast genome in one copy (haploid strains) or two copies (diploid strains).

MazF and MazE are expressed from different types of promoters. First, MazF is cloned under control of the inducible GAL1-GAL10 promoter, which provides a sharp induction of expression when the strain grows on media with raffinose as a single source of carbon. Addition of galactose to the media results in sharp increase of mRNA production within 15-20 minutes from induction. Increase in expression is monitored by S1 ribonuclease assays (see Nikolskaya, et al. (1999)).

S. cerevisiae strains with a GAL80 deletion are used, which provides constitutive expression from the GAL1-GAL10 promoter in up to 10 gene copy numbers (strains GTY106 and GTY107, obtained from Dr. Esposito's lab, University of Chicago). In a related experiment, the same S. cerevisiae strain is employed using the high copy number, non-integrative plasmid pRS426, whose origin of replication provides 20-50 copies of the cloned gene.

### Example 11

### Growth of a Mutant S. cerevisiae Strain Constitutively Expressing MazF Under Control of a Weak Constitutive Promoter and not Expressing MazE

MazF is expressed in a Saccharomyces cerevisiae host strain from a weak constitutive promoter. MazE is not expressed due to a genomic deletion. The mutant S. cerevisiae strain weakly expressing MazF is grown as a batch culture on the YPD medium under the growth conditions as described in Example 9. The culture grows exponentially with a maximum growth rate (μₘₐₓ) of about 0.05 h⁻¹.

### Example 12

### Growth of a Mutant S. cerevisiae Strain Constitutively Expressing MazF Under Control of a Weak Constitutive Promoter and Expressing MazE Under Control of an Inducible Promoter

MazF is expressed in a Saccharomyces cerevisiae host strain from a weak constitutive promoter. MazE is expressed from an inducible promoter, such that the induction of MazE expression coincides with the start of fermentation. The mutant S. cerevisiae is grown as a batch culture on the YPD medium under the growth conditions as described in Example 9. The culture grows exponentially with a maximum growth rate (μₘₐₓ) of about 0.31 h⁻¹.

The above examples are included for illustrative purposes only and are not intended to limit the scope of the invention. Many variations to the example described above are possible. Since modifications and variations to the example described above will be apparent to those of skill in this art, it is intended that this invention be limited only by the scope of the appended claims.

### REFERENCES

Aizenman E, Engelberg-Kulka H, Glaser G (1996) An Escherichia coli chromosomal ‘addiction module’ regulated by guanosine 3′5′-bispyrophosphate: a model for programmed bacterial cell death. Proc. Natl. Acad. Sci. USA 93: 6059-6063.

Christensen S K, Pedersen K, Hansen F G, Gerdes K (2003) Toxin-antitoxin loci as stress-response-elements: ChpAK/MazF and ChpBK cleave translated RNAs and are counteracted by tmRNA. J. Mol. Biol. 332: 809-819.

Condon C (2006) Shutdown decay of mRNA. Mol. Microbiol. 61: 573-583.

Datsenko K., Wanner B. L. One-step inactivation of chromosomal genes in Escherichia coli K-12 using PCR products. Proc. Natl. Acad. Sci., (2000), 97(12): 6640-6645.

Engelberg-Kulka H, Sat B, Reches M, Amitai S, Hazan R (2004) Bacterial programmed cell death systems as targets for antibiotics. Trends Microbiol. 12: 66-71.

Epshtein V. & Nudler E. (2005) Cooperation between RNA polymerase molecules in transcription elongation. Science, 300: 801-805.

Gerdes K, Christensen S K, Lobner-Olesen A (2005) Prokaryotic toxin-antitoxin stress-response loci. Nat. Rev. Microbiol. 3: 371-382.

Hazan R, Sat B, Engelberg-Kulka H (2004) Escherichia coli mazEF-mediated cell death is triggered by various stressful conditions. J. Bacteriol. 186: 3663-3669.

Hensing M C M, Rouwenhorst R J, Heijen J J, van Dijken J P, Pronk J T (1995) Physiological and technical aspects of large-scale heterologous-protein production with yeasts. Ant. van Leeuwenhoek 67: 261-279.

Inouye M (2006) The discovery of mRNA interferases: implication in bacterial physiology and application to biotechnology. J. Cell. Physiol. 209: 670-676.

Inouye M, Zhang J, Zhang Y L (2008) RNA interferases and methods of use thereof. U.S. Patent Pub. No. 2008/0058275.

Lee S Y (1996) High cell-density culture of Escherichia coli. Trends Biotechnol. 14: 98-105.

Marianovsky I, Aizenman E, Engelberg-Kulka H, Glaser G (2001) The regulation of the Escherichia coli mazEF promoter involves an unusual alternating palindrome. J. Biol. Chem. 276: 5975-5984.

Mattanovich D, Gasser B, Hohenblum S, Sauer M (2004) Stress in recombinant protein producing yeasts. J. Biotechnol. 113: 121-135.

Riezenberg D and Guthke R (1999) High-cell-density cultivation of microorganisms. Appl. Microbiol. Biotechnol. 51: 422-430.

Ruiz-Echevarria M J, Benzal-Herranz A, Gerdes K, Diaz-Orejas R (1991) The kis and kid genes of the parD maintenance sys tem of plasmid R1 form an operon that is autoregulated at the level of transcription by the co-ordinanted action of the Kis and Kid proteins. Mol. Microbiol 5: 2685-2693.

Sanden A M, Prytz I, Tubulekas I, F\check{r}berg C, Le H, Hektor A, Neubauer P, Pragai Z, Harwood C, Ward A, Pikon A, Teixeira de Matos J, Postma P, Farewell A, Nystr\check{o}m T, Reeh S. Pedersen S, Larsson G (2003) Limiting factors in Escherichia coli fed-batch production of recombinant proteins. Biotechnol. Bioeng. 81: 158-166.

Sat B, Hazan R, Fisher T, Khaner H, Glaser G, Engelberg-Kulka H (2001) Programmed cell death of Escherichia coli: some antibiotics can trigger mazEF lethality. J Bacteriol. 183: 2041-2045.

Sat B, Reaches M, Engelberg-Kulka H (2003) The Escherichia coli mazEF suicide module mediates thymineless death. J. Bacteriol. 185: 1803-1807.

Suzuki M, Zhang J, Liu M, Woychik NA, Inouye M (2005) Single protein production in living cells facilitated by an mRNA interferase. Mol. Cell 18: 253-261.

Teich A, Meyer S, Lin H Y, Anderson L, Enfors S-O, Neubauer P (1999) Biotechnol. Prog. 15: 123-129.

Tsucimoto S, Ohtsubo E (1993) Autoregulation by cooperative binding of the PemI and PemK proteins to the promoter region of the pem operon. Mol. Gen. Genet. 237: 81-88.

Zhang J, Zhang Y, Inouye M (2003a) Characterization of the interactions within the mazEF addiction module of Escherichia coli. J. Biol. Chem. 278: 32300-32306.

Zhang Y, Zhang J, Hoeflich K P, Ikura M, Quing G, Inouye M (2003b) MazF cleaves cellular mRMAs specifically at ACA to block protein synthesis in Escherichia coli. Mol. Cell 12: 913-923.

Zhang Y, Zhang J, Hara H, Kato I, Inouye M (2005) Insights into the mRNA cleavage mechanism by MazF, an mRNA interferase. J. Biol. Chem. 280: 3143-3150.

All publications and patent applications cited in this specification are herein incorporated by reference as if each individual publication or patent application were specifically and individually indicated to be incorporated by reference.

1. A system for decreasing a cellular growth rate, said system comprising a host cell comprising a first nucleotide sequence encoding an mRNA interferase operably linked to a first heterologous regulatory element, wherein the expression of said first nucleotide sequence encoding mRNA interferase in said host cell diminishes said growth rate but does not arrest cellular growth completely.

2. The system of claim 1, further comprising a second nucleotide sequence encoding an antitoxin protein cognate to said mRNA interferase, wherein said cognate antitoxin protein is operably linked to a second heterologous regulatory element, and said second heterologous regulatory element is different from said first heterologous regulatory element.

3. The system of claim 1 or 2, wherein said first heterologous regulatory element is a weak constitutive promoter.

4. The system of claim 1 or 2, wherein said first heterologous regulatory element is an inducible promoter.

5. The system of claim 4, wherein said inducible promoter is responsive to isopropyl β-D-1-thiogalactopyranoside (IPTG).

6. The system of claim 2, wherein said second heterologous regulatory element is a weak constitutive promoter.

7. The system of claim 2, wherein said second heterologous regulatory element is an inducible promoter.

8. The system of claim 7, wherein said inducible promoter is responsive to isopropyl β-D-1-thiogalactopyranoside (IPTG).

9. The system of claim 1 or 2, wherein said mRNA interferase is selected from the group consisting of Escherichia coli (E. coli) MazF, E. coli ChpBK, E. coli PemK and Bacillus subtilis YdcE.

10. The system of claim 2, wherein said cognate antitoxin protein is selected from the group consisting of Escherichia coli (E. coli) MazE, E. coli ChpBI, E. coli PemI, and Bacillus subtilis YdcD.

11. A method for decreasing a cellular growth rate, comprising the steps of:
(a) providing a host cell;
(b) cloning a first nucleotide sequence encoding an mRNA interferase;
(c) operably linking said first nucleotide sequence encoding mRNA interferase to a first heterologous regulatory element; and
(d) expressing said first nucleotide sequence encoding mRNA interferase operably linked to said first heterologous regulatory element in said host cell,
wherein the expression of said first nucleotide sequence encoding mRNA interferase in said host cell diminishes said cellular growth rate but does not arrest cellular growth completely.

12. The method of claim 11, further comprising the steps of:
(e) cloning a nucleotide sequence encoding a second nucleotide sequence encoding an antitoxin protein cognate to said mRNA interferase;
(f) operably linking said second nucleotide sequence encoding said cognate antitoxin to a second heterologous regulatory element; and
(g) expressing said second nucleotide sequence encoding said cognate antitoxin operably linked to said second heterologous regulatory element in said host cell,
wherein said second heterologous regulatory element is different from said first heterologous regulatory element.

13. The method of claim 11 or 12, wherein said first heterologous regulatory element is a weak constitutive promoter.

14. The method of claim 11 or 12, wherein said first heterologous regulatory element is an inducible promoter.

15. The method of claim 14, wherein said inducible promoter is responsive to isopropyl β-D-1-thiogalactopyranoside (IPTG).

16. The system of claim 12, wherein said second heterologous regulatory element is a weak constitutive promoter.

17. The method of claim 12, wherein said second heterologous regulatory element is an inducible promoter.

18. The method of claim 17, wherein said inducible promoter is responsive to isopropyl β-D-1-thiogalactopyranoside (IPTG).

19. The method of claim 11 or 12, wherein said mRNA interferase is selected from the group consisting of Escherichia coli (E. coli) MazF, E. coli ChpBK, E. coli PemK and Bacillus subtilis YdcE.

20. The method of claim 12, wherein said cognate antitoxin protein is selected from the group consisting of Escherichia coli (E. coli) MazE, E. coli ChpBI, E. coli PemI, and Bacillus subtilis YdcD.

21. A method for decreasing accumulation of toxic metabolites during fermentation, comprising the steps of:
(a) providing a host cell;
(b) cloning a first nucleotide sequence encoding an mRNA interferase;
(c) operably linking said first nucleotide sequence encoding mRNA interferase to a first heterologous regulatory element; and
(d) expressing said first nucleotide sequence encoding mRNA interferase operably linked to said first heterologous regulatory element in said host cell,
wherein the expression of said first nucleotide sequence encoding mRNA interferase in said host cell diminishes accumulation of said toxic metabolites during said fermentation.

22. The method of claim 21, further comprising the steps of:
(e) cloning a nucleotide sequence encoding a second nucleotide sequence encoding an antitoxin protein cognate to said mRNA interferase;
(f) operably linking said second nucleotide sequence encoding said cognate antitoxin to a second heterologous regulatory element; and
(g) expressing said second nucleotide sequence encoding said cognate antitoxin operably linked to said second heterologous regulatory element in said host cell,
wherein said second heterologous regulatory element is different from said first heterologous regulatory element.

23. The method of claim 21 or 22, wherein said first heterologous regulatory element is a weak constitutive promoter.

24. The method of claim 21 or 22, wherein said first heterologous regulatory element is an inducible promoter.

25. The method of claim 24, wherein said inducible promoter is responsive to isopropyl β-D-1-thiogalactopyranoside (IPTG).

26. The system of claim 22, wherein said second heterologous regulatory element is a weak constitutive promoter.

27. The method of claim 22, wherein said second heterologous regulatory element is an inducible promoter.

28. The method of claim 27, wherein said inducible promoter is responsive to isopropyl β-D-1-thiogalactopyranoside (IPTG).

29. The method of claim 21 or 22, wherein said mRNA interferase is selected from the group consisting of Escherichia coli (E. coli) MazF, E. coli ChpBK, E. coli PemK and Bacillus subtilis YdcE.

30. The method of claim 22, wherein said cognate antitoxin protein is selected from the group consisting of Escherichia coli (E. coli) MazE, E. coli ChpBI, E. coli PemI, and Bacillus subtilis YdcD.

31. A method for decreasing oxygen consumption during fermentation, comprising the steps of:
(a) providing a host cell;
(b) cloning a first nucleotide sequence encoding an mRNA interferase;
(c) operably linking said first nucleotide sequence encoding mRNA interferase to a first heterologous regulatory element; and
(d) expressing said first nucleotide sequence encoding mRNA interferase operably linked to said first heterologous regulatory element in said host cell,
wherein the expression of said first nucleotide sequence encoding mRNA interferase in said host cell diminishes said oxygen consumption during said fermentation.

32. The method of claim 31, further comprising the steps of:
(e) cloning a nucleotide sequence encoding a second nucleotide sequence encoding an antitoxin protein cognate to said mRNA interferase;
(f) operably linking said second nucleotide sequence encoding said cognate antitoxin to a second heterologous regulatory element; and
(g) expressing said second nucleotide sequence encoding said cognate antitoxin operably linked to said second heterologous regulatory element in said host cell,
wherein said second heterologous regulatory element is different from said first heterologous regulatory element.

33. The method of claim 31 or 32, wherein said first heterologous regulatory element is a weak constitutive promoter.

34. The method of claim 31 or 32, wherein said first heterologous regulatory element is an inducible promoter.

35. The method of claim 34, wherein said inducible promoter is responsive to isopropyl β-D-1-thiogalactopyranoside (IPTG).

36. The system of claim 32, wherein said second heterologous regulatory element is a weak constitutive promoter.

37. The method of claim 32, wherein said second heterologous regulatory element is an inducible promoter.

38. The method of claim 37, wherein said inducible promoter is responsive to isopropyl β-D-1-thiogalactopyranoside (IPTG).

39. The method of claim 31 or 32, wherein said mRNA interferase is selected from the group consisting of Escherichia coli (E. coli) MazF, E. coli ChpBK, E. coli PemK and Bacillus subtilis YdcE.

40. The method of claim 32, wherein said cognate antitoxin protein is selected from the group consisting of Escherichia coli (E. coli) MazE, E. coli ChpBI, E. coli PemI, and Bacillus subtilis YdcD.

41. A method for decreasing heat generation during fermentation, comprising the steps of:
(a) providing a host cell;
(b) cloning a first nucleotide sequence encoding an mRNA interferase;
(c) operably linking said first nucleotide sequence encoding mRNA interferase to a first heterologous regulatory element; and
(d) expressing said first nucleotide sequence encoding mRNA interferase operably linked to said first heterologous regulatory element in said host cell,
wherein the expression of said first nucleotide sequence encoding mRNA interferase in said host cell diminishes said heat generation during said fermentation.

42. The method of claim 41, further comprising the steps of:
(e) cloning a nucleotide sequence encoding a second nucleotide sequence encoding an antitoxin protein cognate to said mRNA interferase;
(f) operably linking said second nucleotide sequence encoding said cognate antitoxin to a second heterologous regulatory element; and
(g) expressing said second nucleotide sequence encoding said cognate antitoxin operably linked to said second heterologous regulatory element in said host cell,
wherein said second heterologous regulatory element is different from said first heterologous regulatory element.

43. The method of claim 41 or 42, wherein said first heterologous regulatory element is a weak constitutive promoter.

44. The method of claim 41 or 42, wherein said first heterologous regulatory element is an inducible promoter.

45. The method of claim 44, wherein said inducible promoter is responsive to isopropyl β-D-1-thiogalactopyranoside (IPTG).

46. The system of claim 42, wherein said second heterologous regulatory element is a weak constitutive promoter.

47. The method of claim 42, wherein said second heterologous regulatory element is an inducible promoter.

48. The method of claim 47, wherein said inducible promoter is responsive to isopropyl β-D-1-thiogalactopyranoside (IPTG).

49. The method of claim 41 or 42, wherein said mRNA interferase is selected from the group consisting of Escherichia coli (E. coli) MazF, E. coli ChpBK, E. coli PemK and Bacillus subtilis YdcE.

50. The method of claim 42, wherein said cognate antitoxin protein is selected from the group consisting of Escherichia coli (E. coli) MazE, E. coli ChpBI, E. coli PemI, and Bacillus subtilis YdcD.

51. The method of claim 11 or 12, further comprising the steps of:
modifying an endogenous or heterologous gene of interest to substitute one or more mRNA nucleotide recognition sequence with a nucleotide sequence that is not cleavable by said mRNA interferase being expressed, wherein the amino acid sequence of the protein encoded by said gene of interest is not altered; and
co-expressing said gene of interest in said host cell.

52. The method of claim 51, wherein said mRNA interferase is MazF and said mRNA recognition nucleotide sequence is ACA.
